(19) United States
(12) Patent Application Publication
(10) Pub. No.: US 2018/0116925 A1
(43) Pub. Date: May 3, 2018

(54) SUNSCREEN COMPOSITIONS

(71) Applicant: Australian Gold, LLC, Indianapolis, IN (US)

(72) Inventors: Maxine Johnson, Arizona City, AZ (US); Angie H. Provo, Carmel, IN (US); Hillary Leonora Rebollar, Westfield, IN (US); Pamela Johnson, Whitestown, IN (US); Kristen Steinbrook, Indianapolis, IN (US); Brookelynn Liegibel, Franklin, IN (US)

(73) Assignee: Australian Gold, LLC, Indianapolis, IN (US)

(21) Appl. No.: 15/797,321

(22) Filed: Oct. 30, 2017

Related U.S. Application Data
(60) Provisional application No. 62/496,856, filed on Oct. 31, 2016.

(51) Int. Cl. A61K 8/29; A61K 8/27; A61K 8/891; A61K 8/06; A61Q 17/04

(52) U.S. Cl. CPC A61K 8/29; A61K 8/27; A61Q 17/04; A61K 8/064; A61K 8/891

(57) ABSTRACT

The present disclosure relates to compositions which provide UV protection to the skin when applied. In certain embodiments, the present disclosure comprises one or more mineral sunscreens formulas.

### CROSS-REFERENCE TO RELATED APPLICATION

This application claims the benefit of U.S. Provisional Application No. 62/496,856 filed Oct. 31, 2016, which is hereby incorporated by reference in its entirety.

### FIELD OF THE INVENTION

The present disclosure relates to improved sunscreen compositions.

### BACKGROUND

Ultraviolet radiation (“UV”) is the name given to the portion of the electromagnetic radiation that occurs at wavelengths shorter than visible light. The wavelengths that comprise the UV spectrum are typically understood to be between about 400 nm and about 290 nm. Sunlight comprises two UV regions, the UVA region and the UVB region. UVA is generally understood to be between about 400 nm and about 320 nm, while UVB is generally understood to be between about 320 nm and about 290 nm. Because the depth that electromagnetic waves penetrate substrates, e.g., skin, is related to the wavelength of the electromagnetic radiation, UVA radiation penetrates deeper than UVB radiation.

Both UVA and UVB rays can cause tanning of the skin and overexposure can cause reddening and/or potential skin damage. Sunscreen compositions can be used to reduce the skin's exposure to UVA and UVB radiation. Sunscreen compositions that block UVA and UVB radiation are typically called “wide-spectrum” sunscreens.

To measure the reduction in transmission of UV radiation by a composition, the “sun protection factor” or “SPF” is often used. The SPF of a composition is the inverse of the fraction of UV rays that reach the skin. It is common for dermatologists to recommend compositions with SPF values of greater than or equal to about 15.

There are two basic approaches to formulating the active ingredients in a sunscreen. In one approach, “natural” or “mineral” sunscreen compositions comprise active ingredients of titanium dioxide and zinc oxide. These inorganic minerals form a physical barrier and reflect UV radiation before it reaches the skin. In the other approach, “chemical sunscreens” include, but are not limited to, active ingredients such as oxybenzone, octinoxate, octisalate and/or avobenzone. Chemical sunscreens are absorbed by the skin and reduce UV exposure of the skin by absorbing the UV radiation and converting this energy into heat or other forms of energy. There are various pros and cons to each approach. For example, natural sunscreens are less oily, are harder to wash off, and are believed to be less allergenic, yet mineral compositions are often difficult to effectively spread uniformly on the skin. Chemical sunscreens, in comparison, are easier to spread for uniform coverage and may be easier to formulate and mix with other ingredients.

There remains a need for sunscreen compositions, including sunscreen compositions with improved rheological properties.

### BRIEF DESCRIPTION OF THE DRAWINGS

FIG. 1 shows a digital image of Panelist A's arm with two compositions applied side-by-side and after initial application.

FIG. 2 shows a digital image of Panelist A's arm with two compositions applied side-by-side and spread for use.

FIG. 3 shows a digital image of Panelist B's arm with two compositions applied side-by-side and after initial application.

FIG. 4 shows a digital image of Panelist B's arm with two compositions applied side-by-side and spread for use.

FIG. 5 shows a digital image of Panelist C's arm with two compositions applied side-by-side and after initial application.

FIG. 6 shows a digital image of Panelist C's arm with two compositions applied side-by-side and spread for use.

FIG. 7 shows a digital image of two compositions applied to the arms of Panelists A, Panelist B, and Panelist C.

### SUMMARY

In certain embodiments, the present disclosure comprises mineral sunscreen compositions formulated as water in oil emulsions with silicon-containing polymers present. The compositions do not exhibit phase inversion when applied to the skin, allowing the compositions to be more easily spread uniformly on the skin. The formulations encourage more efficient and effective use of the sunscreen by consumers, translating to a lower volume of sunscreen used by the consumer to obtain a desired SPF protection. In example embodiments, a low concentration of active mineral sunscreen ingredients can be used to achieve a surprisingly high SPF efficacy.

Compositions of the present disclosure exhibit surprising results in that a lower concentration of natural sunscreens is needed in the composition to observe similar sun protection factors. Additionally, compositions of the present disclosure may exhibit improved rheological properties, including, but not limited to easier application as the compositions are spread over the skin.

Selected embodiments may comprise pigments of iron oxide and/or mixtures of iron oxide. The combination helps reduce undesired whiteness or chalkiness that is often associated with mineral sunscreens. The resulting combination may instead apply a cosmetic tint in a selected color and hue to the skin, as determined by the pigment added.

Additional embodiments of the invention, as well as features and advantages thereof, will be apparent from the descriptions herein.

### DETAILED DESCRIPTION

For the purposes of promoting an understanding of the principles of the disclosure, reference will now be made to certain embodiments and specific language will be used to describe the same. It will nevertheless be understood that no limitation of the scope of the disclosure is thereby intended, such alterations and further modifications, and such further applications of the principles of the disclosure as described herein being contemplated as would normally occur to one skilled in the art to which the disclosure relates. Additionally, in the detailed description below, numerous alternatives are given for various features. It will be understood that each such disclosed alternative, or combinations of such alternatives, can be combined with the more generalized features discussed in the Summary above, or set forth in the embodiments described below to provide additional disclosed embodiments herein.

In certain embodiments, the present disclosure comprises mineral sunscreen compositions formulated as water in oil emulsions with silicon-containing polymers present. The compositions do not exhibit phase inversion when applied to the skin, allowing the compositions to be more easily spread uniformly on the skin. The formulations encourage more efficient and effective use of the sunscreen by consumers translating to a lower volume of sunscreen used by the consumer to obtain a desired SPF protection. In example embodiments, a low concentration of active mineral sunscreen ingredients can be used to achieve a surprisingly high SPF efficacy.

Compositions of the present disclosure exhibit surprising results in that a lower concentration of natural sunscreens is needed in the composition to observe similar sun protection factors. For example, as typically employed, when 5% (w/w) TiO₂ is used, an SPF value of 3.2 is observed and when 5% (w/w) ZnO is used, an SPF of 6 is observed. As will be discussed below, in embodiments of the present disclosure where about 4% (w/w) TiO₂ and about 4% ZnO (w/w) are used, an SPF value of 50 is observed. This high level of protection from the sun, observed while using comparatively less sunscreen ingredients (10% versus 8% total TiO₂ and ZnO) in the formulation is surprising and particularly efficacious. Other examples of the typical sun protection typically observed can be found in certain product literature relating to natural sunscreens. For example, product literature from Vizor indicates that for ultrafine uncoated ZnO 1.3 to 1.7 SPF units can be observed for 1% of the ZnO ingredient. (Vizor product literature, http://vizorsun.com/super-zinc-1000/). Other examples include that for Super Zinc® 1000 3.0 to 3.3 SPF units can be observed for 1% of the ZnO ingredient in Super Zinc® 1000. (Vizor product literature, http://vizorsun.com/super-zinc-1000/). From these data in the product literature, it can be calculated that for ultrafine uncoated ZnO alone to achieve an SPF of 50, almost 30% ultrafine uncoated ZnO would be needed, and for Super Zinc® alone to achieve an SPF of 50, 15% SuperZinc® would be needed. Embodiments of the present disclosure, for example, Example 1, when 4% of ZnO and 4% of TiO2 are used, an SPF of 50 is observed for the formulation. This unexpectedly low concentration of natural sunscreens provides an SPF of 50 in the formulation of Example 1.

Additionally, compositions of the present disclosure may exhibit improved rheological properties, including, but not limited to easier application and/or by generating less friction as the compositions are spread over the skin.

Compositions of the present disclosure may comprise low levels of one or more natural and/or mineral sunscreen agents. Natural and/or mineral sunscreen agents include, but are not limited to, titanium dioxide (TiO₂), and zinc oxide (ZnO). In certain embodiments one or both of titanium dioxide and zinc oxide may be present in a combined total concentration up to about 10% by weight of the total composition. In certain embodiments one or both of titanium dioxide and zinc oxide may be present in a combined total concentration of 8% or less by weight of the total composition. In certain other embodiments one or both of titanium dioxide and zinc oxide may be present in a combined total concentration of 6% or less by weight of the total composition.

In certain embodiments, titanium dioxide (TiO₂) may be present in a concentration between about 1% by weight and about 5% by weight. In certain embodiments, zinc oxide (ZnO) may be present in a concentration between about 1% by weight and about 5% by weight. In certain embodiments, titanium dioxide is present in a concentration less than about 4% by weight, either alone or in combination with zinc oxide. In certain other embodiments, zinc oxide present in a concentration less than about 4% by weight, either alone or in combination with titanium dioxide. For example, in certain embodiments with 4% titanium dioxide and 4% ZnO, an SPF of 50 is achieved. In alternate embodiments, titanium dioxide and zinc oxide are each present in a concentration less than about 3% by weight, either alone or in combination. For example, in certain embodiments with 3% titanium dioxide and 3% ZnO, an SPF of 30 is achieved. In selected embodiments, the titanium dioxide and/or zinc oxide particles have diameters that are in the micrometer range.

Some embodiments may comprise pigments of iron oxide and/or mixtures of iron oxide. Traditionally, mineral sunscreens with high concentrations of active ingredients are visible with a whitish color when applied to the skin. When iron oxide and/or iron oxides are used with the mineral sunscreen ingredients, the combination results in a solution which eliminates, reduces, and/or masks any whiteness when applied. The resulting combination may become virtually invisible on the skin or may instead apply a cosmetic tint in a selected color and hue to the skin, as determined by the amounts and proportions of pigment used.

In applying a sunscreen, a consumer typically applies a quantity of the sunscreen to the skin, for example by pouring or spraying a quantity directly onto exposed skin or by pouring a quantity into their palm or onto their fingers and then transferring the quantity to the skin surface. Typically, the consumer manually spreads the sunscreen across the skin with a rubbing motion, attempting to achieve uniform coverage. Traditional sunscreens with high concentrations of mineral sunscreen ingredients have a higher friction coefficient on the skin, making the sunscreen more difficult to spread. This can lead to a consumer not effectively spreading the sunscreen and, for example, missing areas which are desired to be covered. Alternately, a consumer may use an excess amount of sunscreen to ensure complete coverage, but which may create waste and inefficiency. An excess amount applied can also leave a whitish color on the skin, which maybe undesired.

A novel aspect of the present composition is increased ease for a consumer to uniformly spread the composition on the skin. The compositions are water in oil emulsions and do not exhibit phase inversion when applied to the skin. This allows the compositions to be more easily spread uniformly on the skin. The formulations encourage more efficient and effective use of the sunscreen by consumers, translating to a lower volume of sunscreen used by the consumer. Achieving uniform coverage more efficiently also allows the composition to provide a desired SPF protection level with a lower level of mineral active ingredients.

Compositions of the present disclosure may comprise preservatives, one or more antioxidants, one or more pH modifiers, one or more pH buffer systems, one or more fragrances, one or more thickening agents, one or more emulsifying agents, one or more antifungal agents, one or more antimicrobial agents, one or more humectants, one or more emollients, one or more surfactants, one or more sunscreens, and/or one or more solvents. The total quantity of any one or more additives may be any suitable quantity as would be employed by those of ordinary skill in the art.

Various moisturizing agents or humectants that may be included in embodiments of the compositions of the present disclosure include, but are not limited to, amino acids, chondroitin sulfate, diglycerin, erythritol, fructose, glucose, glycerin, glycerol polymers, glycol, dimethyl isosorbide, 1,2,6-hexanetriol, honey, hyaluronic acid, hydrogenated honey, hydrogenated starch hydrolysate, inositol, lactitol, maltitol, maltose, mannitol, natural moisturization factor, PEG-15 butanediol, polyglyceryl sorbitol, salts of pyrollidone carboxylic acid, potassium PCA, propylene glycol, sodium glucuronate, sodium PCA, sorbitol, sucrose, trehalose, urea, and xylitol, hydrogenated castor oil, PEG-40 hydrogenated castor oil, PEG-20 methyl glucose sesquistearate, PEG-40 sorbitan peroleate, PEG-5 soy sterol, PEG-10 soy sterol, PEG-2 stearate, PEG-8 stearate, PEG-20 stearate, PEG-32 stearate, PEG-40 stearate, PEG-50 stearate, PEG-100 stearate, PEG-150 stearate, pentadecalactone, peppermint (mentha piperita) oil, petrolatum, phospholipids, polyamino sugar condensate, polyglyceryl-3 diisostearate, polyquaternium-24, polysorbate 20, polysorbate 40, polysorbate 60, polysorbate 80, polysorbate 85, potassium myristate, potassium palmitate, potassium sorbate, potassium stearate, propylene glycol, propylene glycol dicaprylate/dicaprate, propylene glycol dioctanoate, propylene glycol dipelargonate, propylene glycol laurate, propylene glycol stearate, propylene glycol stearate SE, PVP, pyridoxine dipalmitate, quaternium-15, quaternium-18 hectorite, quaternium-22, retinol, retinyl palmitate, rice (oryza sativa) bran oil, RNA, rose oil, safflower (carthamus tinctorius) oil, sage (salvia officinalis) oil, salicylic acid, sandalwood (santalum album) oil, serine, serum protein, sesame (sesamum indicum) oil, shea butter (butyrospermum parkii), silk powder, sodium chondroitin sulfate, sodium DNA, sodium hyaluronate, sodium lactate, sodium palmitate, sodium PCA, sodium polyglutamate, sodium stearate, soluble collagen, sorbic acid, sorbitan laurate, sorbitan oleate, sorbitan palmitate, sorbitan sesquioleate, sorbitan stearate, sorbitol, soybean (glycine soja) oil, sphingolipids, squalane, squalene, stearamide MEA-stearate, stearic acid, stearoxy dimethicone, stearoxytrimethylsilane, stearyl alcohol, stearyl glycyrrhetinate, stearyl heptanoate, stearyl stearate, sunflower (helianthus annuus) seed oil, sweet almond (prunus amygdalus dulcis) oil, synthetic beeswax, tocopherol, tocopheryl acetate, tocopheryl linoleate, tribehenin, tridecyl neopentanoate, tridecyl stearate, triethanolamine, tristearin, trimethylsiloxysilicate, urea, vegetable oil, water, waxes, wheat (triticum vulgare) germ oil, and ylang ylang (cananga odorata) oil.

Caramel colorants may be used in certain embodiments of the present disclosure. Caramels are often prepared by heating carbohydrates, in the presence of acids, alkalis, or salts. The chemical products of caramel production are varied and can change based upon the processes used to produce the caramel. Caramel colorants are commonly classified in the industry based upon the processes used to produce the caramel. These classes are summarized is below in Table 1.

TABLE 1
Classes of caramel colorants.

| | INS | E | | Restrictions On | |
|---|---|---|---|---|---|
| Class | No. | Number | Description | Preparation | Common uses |
| I | 150a | E150a | Plain caramel, | No ammonium or | Often used in |
| | | | caustic caramel, | sulfite compounds can | whisky or other |
| | | | spirit caramel; | be used during the | high proof |
| | | | | preparation. | alcohols. |
| II | 150b | E150b | Caustic sulfite | Sulfite compounds can | Often used in |
| | | | caramel; | be used but no | cognac, sherry, |
| | | | | ammonium compounds | or vinegars. |
| | | | | can be present | |
| III | 150c | E150c | Ammonia caramel, | ammonium compounds | Often used in |
| | | | baker's caramel, | may be used, but no | beer, sauces, or |
| | | | confectioner's | sulfite compounds may | confections. |
| | | | caramel, beer | be present. | |
| | | | caramel; | | |
| IV | 150d | E150d | Sulfite ammonia | Both ammonium | Often used in |
| | | | caramel, acid-proof | compounds and sulfite | acidic |
| | | | caramel, soft-drink | compounds are present. | environments |
| | | | caramel; | | such as soft |
| | | | | | drinks. |

Two exemplary caramel colorants that may be used in embodiments of the present disclosure include DSL4, a Class IV caramel, and/or SC105, a Class I caramel, both marketed by Sethness Products Company (Skokie, Ill.).

Table 2 summarizes the physical properties of DSL4 and its corresponding physical properties that may be used in various embodiments of the present disclosure. These physical properties include the classification of the caramel, the tinctorial power, the baume of the caramel, the specific gravity of the caramel, the density of the caramel, the pH of the caramel, the typical color intensity of the caramel, and/or the quantity of 4-MEI present in the caramel. Other class IV caramel colorants may be used in certain embodiments based on having one or more similar physical properties, such as a low 4-MEI concentration.

TABLE 2
Summary of physical properties of Class IV caramel colorants
used in certain embodiments of the present disclosure.

| Caramel Colorant | Physical Properties | |
|---|---|---|
| DSL4 (Sethness Products | Class | IV |
| Company of Skokie, IL) | Tinctorial Power, K_{0.56} | 0.370-0.410 |
| | Baume @ 60° F. | 30.7-31.7 |
| | Specific Gravity @ 60° F. | 1.268-1.280 |
| | Pounds per gallon @ 60° F. | 10.56-10.66 |
| | pH | 2.5-3.0 |
| | Typical Color Intensity | 0.230-0.258 |
| | 4-MEI | <30 ppm |

Class IV caramel colorants are produced from carbohydrates that are heated in the presence of both sulfite and ammonium compounds. In certain embodiments, the Class IV caramel colorants may have an isoelectric point between pH 0.5 and 2, and may carry a negative ionic charge above pH 2.

When caramel colorants are used, such caramel colorants will typically have a concentration between greater than about 0% (w/w) and less than or equal to about 15% (w/w). In additional embodiments, when caramel colorants are used, these colorants have a concentration between about 0.05% (w/w) and 13% (w/w). In still other preferred embodiments, when caramel is used as a colorant, the total caramel present is between about 4% (w/w) and about 5% (w/w).

Other colorants may be used in embodiments of the present disclosure. For example, iron oxide and/or mixtures of iron oxides can be used in certain embodiments of the present disclosure. Colorants extracted from plants, such as from beet, rosemary, annatto, saffron, turmeric, turmeric root, purple sweet potato, cochineal, carrots, wheat, corn, pepper, spirulina, chlorophyll, red cabbage, and grape skin may be used in embodiments of the present disclosure. Other colorants are dyes such as, but not limited to, Red 4, Red 33, Red 40, Carmine, Blue 1, Yellow 5, and/or carbon black.

In order to promote a further understanding of the present invention and its various embodiments, the following specific examples are provided. It will be understood that these examples are illustrative and not limiting of the invention.

### Example 1

### Preparation of an Improved Sunscreen Composition

### Materials and Methods:

A composition was prepared according to the concentrations listed in Table 3.

TABLE 3
Concentration of Ingredients of Sunscreen of EXAMPLE 1.

| Ingredient | Concentration (w/w) |
|---|---|
| Titanium Dioxide | 4.0% |
| Zinc Oxide | 4.0% |
| Alumina | 0.3% |
| Shea Butter | 0.1% |
| Caprylyl Glycol | 0.4% |
| Cetyl PEG/PPG-10/1 | 2.5% |
| Dimethicone | |
| Cyclopentasiloxane | 39.5% |
| Dimethicone Crosspolymer | 1% |
| Disodium EDTA | 0.1% |
| Disteardimonium Hectorite | 2.3% |
| Eucalyptus Globulus Leaf | 0.01% |
| Extract | |
| Fragrance | 0.9% |
| Glycerin | 5.1% |
| Hexyl Laurate | 1.3% |
| Panthenol | 0.1% |
| PEG-10 Dimethicone | 1.7% |
| Phenoxyethanol | 0.4% |
| Polyglyceryl-4 Isostearate | 1% |
| Polymethylsilsesquioxane | 2% |
| Porphyra Umbilicalis Extract | trace |
| Silica | 3% |
| Squalane | 0.1% |
| Stearic Acid | 0.4 |
| Terminalia Ferdinandiana | trace |
| (Kakadu Plum) Fruit Extract | |
| Tocopheryl Acetate | 0.2% |
| Water | Remainder |

### Results:

The composition was tested, and the SPF value was calculated to be about 50. Testing included testing to U.S. FDA (2011) and EU standards, including sun protection factor testing, water resistance testing, Broad Spectrum testing, FDA Critical Wavelength testing, COLIPA Water Resistance testing, ISO24442 UVA-PF & Critical Wavelength in-vivo testing.

### Example 2

### Preparation of an Improved Sunscreen Composition

### Materials and Methods:

A composition was prepared according to the concentrations listed in Table 4.

TABLE 4
Concentration of Ingredients of Sunscreen of EXAMPLE 2.

| Ingredient | Concentration (w/w) |
|---|---|
| Titanium Dioxide | 3.0% |
| Zinc Oxide | 3.0% |
| Alumina | 0.2% |
| Shea Butter | 0.1% |
| Caprylyl Glycol | 0.3% |
| Cetyl PEG/PPG-10/1 | 2.5% |
| Dimethicone | |
| Cyclopentasiloxane | 43.2% |
| Dimethicone Crosspolymer | 1.0% |
| Disodium EDTA | 0.1% |
| Disteardimonium Hectorite | 2.3% |
| Eucalyptus Globulus Leaf | 0.01% |
| Extract | |
| Fragrance | 0.9% |
| Glycerin | 5.1% |
| Hexyl Laurate | 1% |
| Panthenol | 0.1% |
| PEG-10 Dimethicone | 1.3% |
| Phenoxyethanol | 0.4% |
| Polyglyceryl-4 Isostearate | 0.8% |
| Polymethylsilsesquioxane | 2.0% |
| Porphyra Umbilicalis Extract | Trace |
| Silica | 3.0% |
| Squalane | 0.1% |
| Stearic Acid | 0.3% |
| Terminalia Ferdinandiana | trace |
| (Kakadu Plum) Fruit Extract | |
| Tocopheryl Acetate | 0.2% |
| Water | Remainder |

### Results:

The composition was tested, and the SPF value was calculated to be about 30.

### Example 3

### Preparation of an Improved Sunscreen Composition

### Materials and Methods:

A tinted composition including iron oxides was prepared according to the concentrations listed in Table 5.

TABLE 5
Concentration of Ingredients of Sunscreen of EXAMPLE 3.

| Ingredient | Concentration (w/w) |
|---|---|
| Titanium Dioxide | 4.0% |
| Zinc Oxide | 4.0% |
| Alumina | 0.3% |
| Shea Butter | 0.1% |
| Caprylyl Glycol | 0.4% |
| Cetyl PEG/PPG-10/1 | 2.5% |
| Dimethicone | |
| Cyclopentasiloxane | 39.5% |
| Dimethicone Crosspolymer | 1% |
| Disodium EDTA | 0.1% |
| Disteardimonium Hectorite | 2.3% |
| Eucalyptus Globulus Leaf | 0.01% |
| Extract | |
| Fragrance | 0.9% |
| Glycerin | 5.1% |
| Hexyl Laurate | 1.3% |
| Iron Oxides | 0.4% |
| Panthenol | 0.1% |
| PEG-10 Dimethicone | 1.7% |
| Phenoxyethanol | 0.4% |
| Polyglyceryl-4 Isostearate | 1% |
| Polymethylsilsesquioxane | 2% |
| Porphyra Umbilicalis Extract | trace |
| Silica | 3% |
| Squalane | 0.1% |
| Stearic Acid | 0.4 |
| Terminalia Ferdinandiana | trace |
| (Kakadu Plum) Fruit Extract | |
| Tocopheryl Acetate | 0.2% |
| Water | Remainder |

An aliquot of the composition according to Table 3 was applied to the arm of three panelists along with a similar composition that did not comprise iron oxides.

### Results:

The composition was tested, and the SPF value was calculated to be about 50.

It was observed that by adding a small quantity of iron oxides that the whiteness that is normally associated with titanium dioxide and/or zinc oxide was markedly reduced. For example, by including the iron oxides a faint whiteness may be perceived after initial application, yet the whiteness disappeared and was virtually invisible after the composition was spread as intended for use. Optionally, the resulting combination may instead apply a cosmetic tint to the skin in a selected color and hue to the skin, as determined by amounts and proportions of pigment used

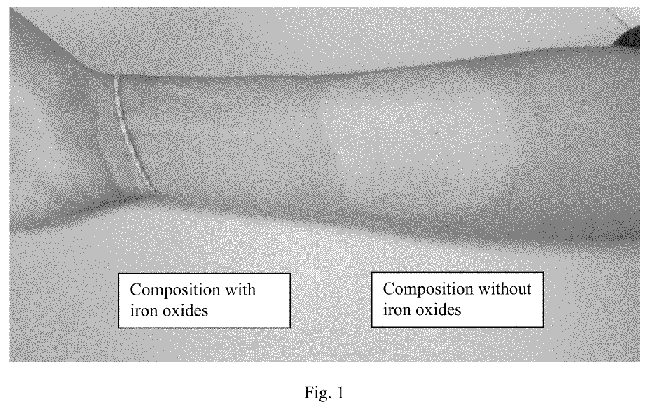

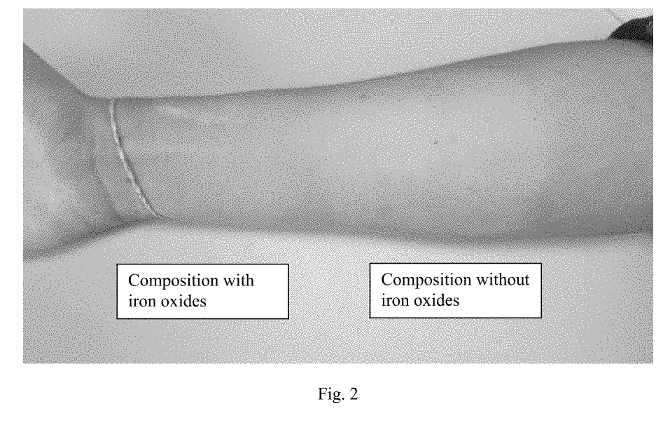

Referring now to the figures, FIG. 1 and FIG. 2 each show a digital image of the forearm is of Panelist A, where a composition according to EXAMPLE 3, a composition comprising iron oxides, was applied to the distal (e.g. elbow) portion of Panelist A's forearm. For comparison a control composition that did not comprise iron oxides was applied to the proximal (e.g. wrist) portion of Panelist A's forearm. FIG. 1 shows the compositions after initial application with a slight amount of spreading having been done as part of the application process. FIG. 2 shows the compositions rubbed in, namely as appropriately spread on the skin for use by a consumer. As can been seen from these digital images, FIG. 1 illustrates that during and after initial application, the composition containing iron oxides had a faint white tint, but substantially less white than the control composition. After the compositions were appropriately spread and rubbed in, FIG. 2 illustrates that the whiteness had disappeared for the composition containing iron oxides.

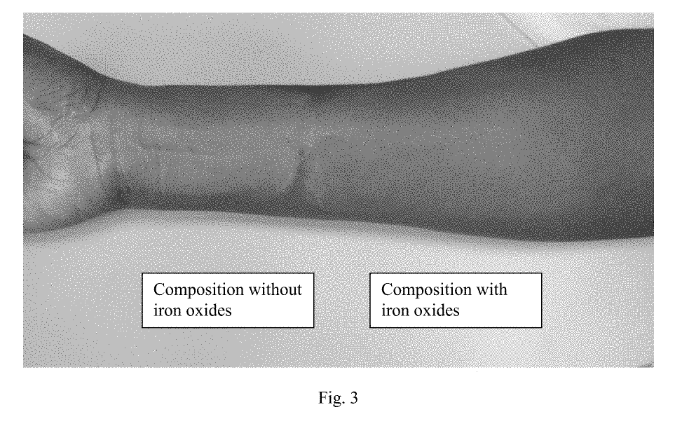

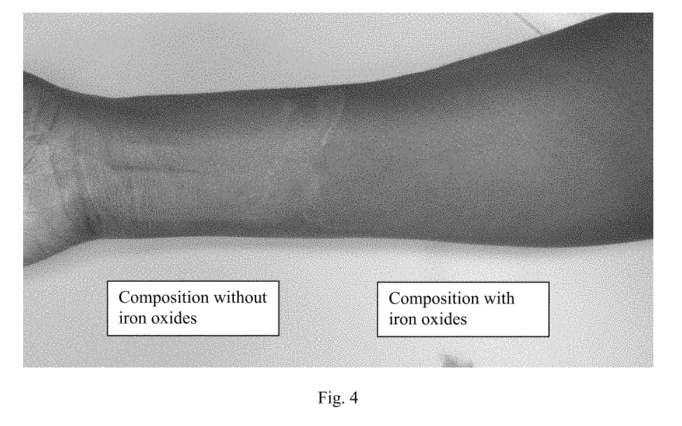

FIG. 3 and FIG. 4 each show a digital image of the forearm of Panelist B, where a composition according to EXAMPLE 3, a composition comprising iron oxides, was applied to the distal (e.g. elbow) portion of Panelist B's forearm. For comparison a composition that did not comprise iron oxides was applied to the proximal (e.g. wrist) portion of Panelist B's forearm. FIG. 3 shows the compositions after initial application with a slight amount of spreading having been done as part of the application process. FIG. 4 shows the compositions rubbed in, namely as appropriately spread on the skin for use by a consumer. As can been seen from these digital images, the composition comprising iron oxides appears less white than the composition that does not comprise iron oxides. As can been seen from these digital images, FIG. 3 illustrates that during and after initial application, the composition containing iron oxides had a faint white tint, but substantially less white than the control composition. After the compositions were appropriately spread and rubbed in, FIG. 4 illustrates that the whiteness had disappeared for the composition containing iron oxides.

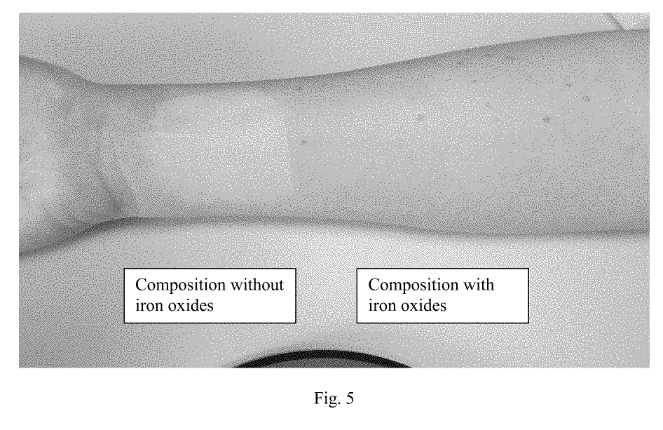

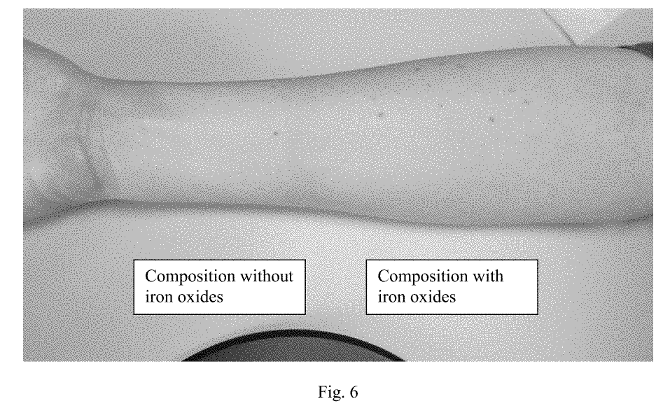

FIG. 5 and FIG. 6 each show a digital image of the forearm of Panelist C, where a composition according to EXAMPLE 3, a composition comprising iron oxides, was applied to the distal (e.g. elbow) portion of Panelist C's forearm. For comparison a composition that did not comprise iron oxides was applied to the proximal (e.g. wrist) portion of Panelist C's forearm. FIG. 5 shows the compositions after initial application with a slight amount of spreading having been done as part of the application process. FIG. 6 shows the compositions rubbed in, namely as appropriately spread on the skin for use by a consumer. As can been seen from these digital images, FIG. 5 illustrates that during and after initial application, the composition containing iron oxides had a faint white tint, but substantially less white than the control composition. After the compositions were appropriately spread and rubbed in, FIG. 6 illustrates that the whiteness had disappeared for the composition containing iron oxides.

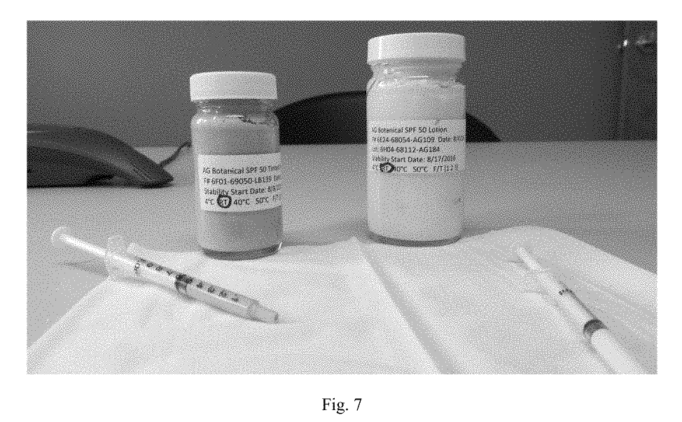

FIG. 7 shows a digital image of the composition of EXAMPLE 3, and a composition that does not comprise iron oxides.

### Example 4

### Material and Methods:

A composition was prepared according to the concentrations listed in Table 6.

TABLE 6
Concentration of Ingredients of Sunscreen of EXAMPLE 3.

| Ingredient | Concentration (w/w) |
|---|---|
| Cyclopentasiloxane | 41.9% |
| Glycerin | 5.1% |
| Hexyl Laurate | 4.1% |
| Silica | 3.0% |
| Zinc Oxide | 3.0% |
| Cetyl PEG/PPG-10/1 | 2.5% |
| Dimethicone | |
| Disteardimonium Hectorite | 2.25% |
| Polymethylsilsesquioxane | 2.0% |
| Titanium Dioxide | 1.75% |
| PEG-10 Dimethicone | 1.1% |
| Dimethicone Crosspolymer | 1.0% |
| Fragrance | 0.83% |
| POlyglyceryl-4 Isostearate | 0.45% |
| Phenoxyethanol | 0.4% |
| Caprylyl Glycol | 0.3% |
| Tocopheryl Acetate | 0.2% |
| Stearic Acid | 0.18% |
| Alumina | 0.13% |
| Panthenol | 0.1% |
| Shea Butter | 0.1% |
| Squalane | 0.1% |
| Disodium EDTA | 0.05% |
| Benzyl Benzoate | 0.03% |
| Kakuda Plum Fruit Extract | 0.005% |
| Porphyra Umbilicalis Extract | 0.005% |
| Eucalyptus Globulus Leaf | 0.005% |
| Extract | |
| Coumarin | 0.004% |
| Linalool | 0.003% |
| Hexyl Cinnamal | 0.003% |
| Benzyl Cinnamate | 0.001% |
| Water | Remainder |

### Results:

The composition of Table 6 was tested, and the SPF value was calculated to be about 15.

The uses of the terms “a” and “an” and “the” and similar references in the context of describing the invention (especially in the context of the following claims) are to be construed to cover both the singular and the plural unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

While the invention has been illustrated and described in detail in the drawings and the foregoing description, the same is to be considered as illustrative and not restrictive in character, it being understood that only the preferred embodiment has been shown and described and that all changes and modifications that come within the spirit of the invention are desired to be protected. In addition, all references cited herein are indicative of the level of skill in the art and are hereby incorporated by reference in their entirety.

What is claimed is:

1. A composition of matter comprising:
about 4% (w/w) titanium dioxide and about 4% (w/w) zinc oxide in an water in oil emulsion having a sun protection value of greater than or equal to about 50.

2. The composition of matter of claim 1, further comprising, one or more silicone polymers.

3. The composition of matter of claim 1, wherein said composition does not exhibit phase inversion when applied to the skin.

4. The composition of matter of claim 1, further comprising iron oxide.

5. The composition of claim 1, further comprising caramel.

6. A composition of matter consisting of, 4.0% (w/w) titanium dioxide, 4.0% (w/w) zinc oxide, 0.3% (w/w) alumina, 0.1% (w/w) shea butter, 0.4% (w/w) caprylyl glycol, 2.5% (w/w) cetyl PEG/PPG-10/1 dimethicone, 39.5% (w/w) cyclopentasiloxane, 1% (w/w) dimethicone crosspolymer, 0.1% (w/w) disodium EDTA, 2.3% (w/w) disteardimonium hectorite, 0.01% (w/w) Eucalyptus Globulus leaf extract, one or more fragrances, 5.1% (w/w) glycerin, 1.3% (w/w) hexyl laurate, 0.1% (w/w) panthenol, 1.7% (w/w) PEG-10 dimethicone, 0.4% (w/w) phenoxyethanol, 1% (w/w) polyglyceryl-4 isostearate, 2% (w/w) polymethylsilsesquioxane, 3% (w/w) silica, 0.1% (w/w) squalane, 0.4 (w/w) stearic acid, one or more fruit or flower extracts, 0.2% (w/w) tocopheryl acetate, and water.

7. A method for reducing ultraviolet radiation from reaching human skin comprising the act of:
topically applying to said human skin the composition of claim 1.

8. A method for increasing the sun protection factor (SPF) of a consumer product comprising one or more silicone polymers relative to a consumer product without a silicone polymer, comprising the acts of:
providing one or more mineral sunscreens;
providing one or more silicone polymers;
providing water; and
creating a water in oil emulsion comprising said one or more mineral sunscreens, said one or more silicone polymers, and said water.

9. The method of claim 8, wherein said one or more mineral sunscreens comprises less than or equal to about 10% (w/w) of the resulting water in oil emulsion.

10. The method of claim 8, wherein said one or more mineral sunscreens comprises less than or equal to about 8% (w/w) of the resulting water in oil emulsion.

11. The method of claim 8, wherein said one or more mineral sunscreens comprises less than or equal to about 6% (w/w) of the resulting water in oil emulsion.

12. The method of claim 8, wherein said one or more mineral sunscreens comprises less than or equal to about 4% (w/w) of the resulting water in oil emulsion.

13. The method of claim 8, wherein said one or more mineral sunscreens comprises titanium dioxide or zinc oxide.

14. The method of claim 8, wherein said mineral sunscreen comprises titanium dioxide.

15. The method of claim 8, wherein said mineral sunscreen comprises zinc oxide.

16. A composition of matter comprising:
about 3% (w/w) titanium dioxide and about 3% (w/w) zinc oxide in an water in oil emulsion having a sun protection value of greater than or equal to about 30.

17. A composition of matter comprising:
about 1.75% (w/w) titanium dioxide and about 3% (w/w) zinc oxide in an water in oil emulsion having a sun protection value of greater than or equal to about 15.
